(19) United States
(12) Patent Application Publication
(10) Pub. No.: US 2017/0274001 A1
(43) Pub. Date: Sep. 28, 2017

(54) Compositions and Methods for Increasing Red Blood Cells

(71) Applicant: Yale University, New Haven, CT (US)

(72) Inventors: Richard Flavell, Guilford, CT (US); Liang Shan, New Haven, CT (US)

(21) Appl. No.: 15/507,151

(22) PCT Filed: Sep. 8, 2015
(86) PCT No.: PCT/US15/48926

§ 371 Date: Feb. 27, 2017

Related U.S. Application Data
(60) Provisional application No. 62/047,341, filed on Sep. 8, 2014.

(51) Int. Cl. A61K 31/713; C12N 15/113; C07K 16/28; A61K 38/17

(52) U.S. Cl. CPC A61K 31/713; A61K 38/1709; C12N 15/1138; C07K 16/2803; A61K 38/179; A01K 2267/0381

(57) ABSTRACT

The invention relates generally to a composition and method for increasing the frequency, amount, or presence of red blood cells in peripheral blood. In one embodiment, the invention comprises the inhibition of, or genetic modification of the genes encoding, one or more scavenger receptors. In one aspect, the invention relates to treatments of anemia.

### CROSS-REFERENCE TO RELATED APPLICATIONS

This application claims priority to U.S. Provisional Application No. 62/047,341 filed Sep. 8, 2014, the contents of which are incorporated by reference herein in their entirety.

### BACKGROUND OF THE INVENTION

The aim of biomedical research is to gain a better understanding of human physiology and to use this knowledge to prevent, treat or cure human diseases. Due to practical and ethical barriers to the experimentation on human subjects, many studies are conducted on small animal models, such as the mouse. However, mice are not people and the knowledge gained from animal experimentation is not always applicable to humans.

Developing a humanized mouse model to support full human erythropoiesis is essential for the study of human erythroid cell-associated diseases, and drug and vaccine discovery. To date no animal models can support human erythropoiesis in the circulation. Although human lymphopoiesis and myelopoiesis have been obtained in various mouse models, no human erythroid lineage cells can be reconstituted, especially in the mouse periphery. The major obstacle is the instantaneous clearance of human erythroid cells in the periphery of heterologous species, including mouse. To date, the mechanism of instantaneous clearance of human erythroid cells in mouse is unknown.

There is a need in the art for compositions and methods to increase red blood cells in the peripheral blood. The present invention addresses this unmet need in the art.

### SUMMARY OF THE INVENTION

In one aspect, the present invention provides a composition for increasing red blood cells (RBCs) in peripheral blood comprising an inhibitor of one or more scavenger receptors. In one embodiment, the inhibitor is an inhibitor of scavenger receptor B1. In one embodiment, the inhibitor is selected from the group consisting of a nucleic acid, a siRNA, an antisense nucleic acid, a ribozyme, a peptide, a small molecule, an antagonist, an aptamer, and a peptidomimetic. In one embodiment, the inhibitor is D-4F. In one embodiment, the inhibitor is a small molecule inhibitor selected from the group consisting of BLT-1 and ITX-5061. In one embodiment, the composition reduces the destruction of red blood cells. In one embodiment, the composition reduces the sequestration of red blood cells.

In one aspect, the present invention provides a method for treating anemia in a subject in need thereof, comprising administering to the subject an effective amount of a composition comprising an inhibitor of one or more scavenger receptors. In one embodiment, the inhibitor is an inhibitor of scavenger receptor B1. In one embodiment, the inhibitor is selected from the group consisting of a nucleic acid, a siRNA, an antisense nucleic acid, a ribozyme, a peptide, a small molecule, an antagonist, an aptamer, and a peptidomimetic. In one embodiment, the inhibitor is D-4F. In one embodiment, the inhibitor is a small molecule inhibitor selected from the group consisting of BLT-1 and ITX-5061. In one embodiment, the method further comprises administering to the subject an effective amount of clodronate.

In one aspect, the present invention provides a method for treating a cholesterol abnormality in a subject in need thereof, comprising administering to the subject an effective amount of a composition comprising an inhibitor of one or more scavenger receptors. In one embodiment, the inhibitor is an inhibitor of scavenger receptor B1. In one embodiment, the inhibitor is selected from the group consisting of a nucleic acid, a siRNA, an antisense nucleic acid, a ribozyme, a peptide, a small molecule, an antagonist, an aptamer, and a peptidomimetic. In one embodiment, the inhibitor is D-4F. In one embodiment, the inhibitor is a small molecule inhibitor selected from the group consisting of BLT-1 and ITX-5061. In one embodiment, the method further comprises administering to the subject an effective amount of clodronate.

In one aspect, the present invention provides a genetically modified non-human animal comprising a genome that is modified such that the animal does not express one or more scavenger receptors. In one embodiment, the one or more scavenger receptors comprise scavenger receptor B1.

In one embodiment, the genetically modified animal comprises at least one of the group consisting of a nucleic acid encoding human EPO, a nucleic acid encoding human M-CSF, a nucleic acid encoding human IL-3, a nucleic acid encoding human GM-CSF, a nucleic acid encoding human SIRPα, and a nucleic acid encoding human TPO, wherein each of the nucleic acids encoding human M-CSF, human IL-3, human GM-CSF, human SIRPα, human TPO and human EPO is operably linked to a promoter, and wherein the animal expresses at least one of the group consisting of human EPO polypeptide, human M-CSF polypeptide, human IL-3 polypeptide, human GM-CSF polypeptide, human SIRPα polypeptide, and human TPO polypeptide.

In one embodiment, the animal is immunodeficient. In one embodiment, the animal does not express recombination activating gene 2 (Rag-2^{-/-}). In one embodiment, the animal does not express IL2 receptor gamma chain (gamma chain^{-/-}). In one embodiment, the animal does not express Rag-2 and wherein the animal does not express IL2 receptor gamma chain (Rag-2^{-/-} gamma chain^{-/-}).

In one embodiment, the animal is a rodent. In one embodiment, the animal is a mouse. In one embodiment, the animal further comprises human hematopoietic cells. In one embodiment, the animal further comprises human erythrocytes.

In one embodiment, the genetically modified animal is infected with malaria. In one embodiment, the animal comprises clodronate. For example, in one embodiment, the animal is treated with clodronate.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts the results of example experiments demonstrating the rapid clearance of human red blood cells in the mouse peripheral blood. Human and mouse red blood cells were pre-labeled with CFSE or violet dye and then were transfused into RGSKI mice by retro-orbital injection. (FIG. 1A) Peripheral blood was collected 5 minutes after blood infusion. Frequency of infused mouse or human RBCs was determined by FACS. (FIG. 1B) Peripheral blood was collected at indicated time points and was analyzed by FACS.

FIG. 2 is a set of graphs demonstrating that human RBCs were rapidly trapped in liver and spleen. Human and mouse red blood cells were pre-labeled with CFSE or violet dye, respectively. After blood infusion, liver, lung, spleen and bone marrow from RGSKI mice were collected at indicated time points.

FIG. 3 is graph depicting the results of experiments demonstrating that Apolipoprotein A-I mimetic peptide D-4F blocks in vivo destruction of human RBCs. D-4F blocks several scavenger receptors. RGSKI mice were treated with different antibodies or inhibitors to block various macrophage receptors. One hour after treatment, mice were infused with human RBCs and peripheral blood was collected at indicated time points. Anti-Dectin: 50 ug/mouse; Anti-CD169: 50 ug/mouse; Anti-CD169 clone 3D6: 100 ug/mice; Anti-Mannose receptor: 50 ug/mouse; D-4F: 10 mg/mouse; Asialofetuin: 10 mg/mouse; Control: PBS.

FIG. 4 is a graph depicting the results of experiments demonstrating that inhibitor of scavenger receptor B1 can protect infused human RBCs in the mouse system. RGSKI mice were treated with scavenger receptor B1 named BLT-1 (80 mg/kg body weight). One hour after treatment, mice were infused with human RBCs and peripheral blood was collected at indicated time points. Control: DMSO.

FIG. 5 is a graph depicting the results of experiments demonstrating that genetic depletion of scavenger receptor B1 can protect infused human RBCs in the mouse system. MISTRG-SRB1^{-/-} or MISTRG-SRB1^{+/-} mice were infused with human RBCs and peripheral blood was collected at indicated time points. Control: MISTRG or MITRG.

FIG. 6 is a graph depicting the results of experiments demonstrating that genetic depletion of scavenger receptor B1 can improve human erythropoiesis and red blood cell survival. MISTERG-SRB1^{+/-} mice were engrafted with human fetal liver CD34⁺ cells. 7 week post engraftment, mice were treated with clodronate and peripheral blood was collected for analysis. Control: MISTERG.

### DETAILED DESCRIPTION

In one embodiment, the present invention relates to compositions and methods for increasing the amount of red blood cells (RBCs) in peripheral blood. The invention is partly based upon the unexpected finding that scavenger receptors play a role in human RBC sequestration and destruction.

In one embodiment, the present invention includes a composition for increasing the frequency, amount, or presence of RBCs in peripheral blood. In one embodiment, the composition comprises an inhibitor of one or more scavenger receptors. In one embodiment, the composition comprises an inhibitor of scavenger receptor B1.

In one embodiment, the present invention includes a method for increasing frequency, amount, or presence of RBCs in peripheral blood. In one embodiment, the method is used to treat certain forms of anemia, including for example sickle cell anemia. In one embodiment, the method is used to treat any cholesterol abnormality associated with red blood cell deformation. In one embodiment, the method comprises administering an inhibitor of one or more scavenger receptors. In one embodiment, the method comprises administering to a subject in need an inhibitor of the scavenger receptor B1. In one embodiment, the method comprises administering an inhibitor of the scavenger receptor B1.

In one embodiment, the present invention includes a non-human animal in which the activity, expression, or both of one or more scavenger receptors are inhibited. In certain instances the inhibition of the one or more scavenger receptors allows for increased human RBCs in the periphery following engraftment of the animal with human cells.

In certain embodiments, the non-human animal is genetically modified to reduce expression or eliminate expression of one or more scavenger receptors. In certain embodiments, the genetically modified non-human animal expresses at least one of human EPO, human M-CSF, human IL-3, human GM-CSF, human SIRPAα, and human TPO. The invention also relates to methods of generating and methods of using the genetically modified non-human animals described herein. In some embodiments, the genetically modified non-human animal is a mouse. In some embodiments, the genetically modified non-human animal described herein is engrafted with human hematopoietic cells. In various embodiments, the human hematopoietic cell engrafted, genetically modified non-human animals of the invention are useful for the in vivo evaluation of the growth and differentiation of hematopoietic and immune cells, for the in vivo evaluation of human hematopoiesis, for the in vivo evaluation of erythropoiesis, for the in vivo evaluation of cancer cells, for the in vivo assessment of an immune response, for the in vivo evaluation of vaccines and vaccination regimens, for the use in testing the effect of agents that modulate cancer cell growth or survival, for the in vivo evaluation of a treatment of cancer, for the in vivo evaluation of malaria infection, for the in vivo evaluation of a treatment of malaria infection, for the in vivo production and collection of immune mediators, including human antibodies, and for use in testing the effect of agents that modulate hematopoietic and immune cell function.

### Definitions

Unless defined otherwise, all technical and scientific terms used herein have the same meaning as commonly understood by one of ordinary skill in the art to which this invention belongs. Such terms are found defined and used in context in various standard references illustratively including Sambrook et al., Molecular Cloning: A Laboratory Manual, Cold Spring Harbor Laboratory Press; 4th Ed., 2012; Ausubel, Ed., Short Protocols in Molecular Biology, Current Protocols; 5th Ed., 2002; Alberts et al., Molecular Biology of the Cell, 4th Ed., Garland, 2002; Nelson and Cox, Lehninger Principles of Biochemistry, 4th Ed., W.H. Freeman & Company, 2004; and Herdewijn (Ed.), Oligonucleotide Synthesis: Methods and Applications, Methods in Molecular Biology, Humana Press, 2004. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “cancer” as used herein is defined as disease characterized by the aberrant proliferation and/or growth of cells. Cancer cells can spread locally or through the bloodstream and lymphatic system to other parts of the body. Cancer as here herein includes both solid tumors and hematopoietic malignancies. Examples of various cancers amenable to the invention include, but are not limited to, breast cancer, prostate cancer, ovarian cancer, cervical cancer, skin cancer, pancreatic cancer, colorectal cancer, renal cancer, liver cancer, bone cancer, brain cancer, lymphoma, leukemia, lung cancer, myeloidysplastic syndromes, myeloproliferative disorders and the like.

“Constitutive” expression is a state in which a gene product is produced in a living cell under most or all physiological conditions of the cell.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a sign or symptom of the disease or disorder, the frequency with which such a sign or symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The terms “expression construct” and “expression cassette” are used herein to refer to a double-stranded recombinant DNA molecule containing a desired nucleic acid human coding sequence and containing one or more regulatory elements necessary or desirable for the expression of the operably linked coding sequence.

As used herein, the term “fragment,” as applied to a nucleic acid or polypeptide, refers to a subsequence of a larger nucleic acid or polypeptide. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between). A “fragment” of a polypeptide can be at least about 15 nucleotides in length; for example, at least about 50 amino acids to about 100 amino acids; at least about 100 to about 500 amino acids, at least about 500 to about 1000 amino acids, at least about 1000 amino acids to about 1500 amino acids; or about 1500 amino acids to about 2500 amino acids; or about 2500 amino acids (and any integer value in between).

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

“Homologous” as used herein, refers to the subunit sequence similarity between two polymeric molecules, e.g. between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g. if half (e.g., five positions in a polymer ten subunits in length) of the positions in two compound sequences are homologous then the two sequences are 50% homologous, if 90% of the positions, e.g. 9 of 10, are matched or homologous, the two sequences share 90% homology. By way of example, the DNA sequences 5′-ATTGCC-3′ and 5′-TATGGC-3′ share 50% homology.

The terms “human hematopoietic stem and progenitor cells” and “human HSPC” as used herein, refer to human self-renewing multipotent hematopoietic stem cells and hematopoietic progenitor cells.

“Inducible” expression is a state in which a gene product is produced in a living cell in response to the presence of a signal in the cell.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “operably linked” as used herein refers to a polynucleotide in functional relationship with a second polynucleotide. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region. Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers both to short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. The term “peptide” typically refers to short polypeptides. The term “protein” typically refers to large polypeptides.

As used herein, a “peptidomimetic” is a compound containing non-peptidic structural elements that is capable of mimicking the biological action of a parent peptide. A peptidomimetic may or may not comprise peptide bonds.

The term “progeny” as used herein refers to a descendent or offspring and includes the differentiated or undifferentiated decedent cell derived from a parent cell. In one usage, the term progeny refers to a descendent cell which is genetically identical to the parent. In another use, the term progeny refers to a descendent cell which is genetically and phenotypically identical to the parent. In yet another usage, the term progeny refers to a descendent cell that has differentiated from the parent cell.

The term “promoter” as used herein refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors. An included promoter can be a constitutive promoter or can provide inducible expression; and can provide ubiquitous, tissue-specific or cell-type specific expression.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A “recombinant polypeptide” is one, which is produced upon expression of a recombinant polynucleotide.

The term “regulatory element” as used herein refers to a nucleotide sequence which controls some aspect of the expression of nucleic acid sequences. Exemplary regulatory elements illustratively include an enhancer, an internal ribosome entry site (IRES), an intron; an origin of replication, a polyadenylation signal (pA), a promoter, an enhancer, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation. Expression constructs can be generated recombinantly or synthetically using well-known methodology.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “transdominant negative mutant gene” refers to a gene encoding a polypeptide or protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Therapeutically effective amount” is an amount of a compound, that when administered to a patient, ameliorates a sign or symptom of a disease or disorder. The amount of a compound of the invention which constitutes a “therapeutically effective amount” will vary depending on the compound, the disorder or disease state and its severity, the age of the patient to be treated, and the like. The therapeutically effective amount can be determined routinely by one of ordinary skill in the art having regard to his own knowledge and to this disclosure.

The terms “subject” or “patient” or “individual” for the purposes of the present invention includes humans and other animals, particularly mammals, and other organisms. Thus the methods are applicable to both human therapy and veterinary applications. In some embodiments the patient is a mammal, and in particular embodiments the patient is human.

The terms “treat,” “treating,” and “treatment,” refer to therapeutic or preventative measures described herein. The methods of “treatment” employ administration to a subject, in need of such treatment, a composition of the present invention, for example, a subject afflicted with a disease or disorder, or a subject who ultimately may acquire such a disease or disorder, in order to prevent, cure, delay, reduce the severity of, or ameliorate one or more signs or symptoms of the disease or disorder or recurring disease or disorder.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, the term “genetically modified” means an animal, the germ cells of which comprise an exogenous human nucleic acid or human nucleic acid sequence. By way of non-limiting examples a genetically modified animal can be a transgenic animal or a knock-in animal, so long as the animal comprises a human nucleic acid sequence.

As used herein, “knock-in” is meant a genetic modification that replaces the genetic information encoded at a chromosomal locus in a non-human animal with a different DNA sequence.

### Description

In one aspect, the present invention relates to compositions and methods for increasing the frequency, amount, or presence of RBCs in peripheral blood. The invention is partly based upon the unexpected finding that scavenger receptors play a role in human RBC sequestration and destruction, and that inhibition of scavenger receptors increases peripheral RBCs.

In one embodiment, the present invention includes a composition for increasing the frequency, amount, or presence of RBCs in peripheral blood. In one embodiment, the composition comprises an inhibitor of one or more scavenger receptors. For example, in one embodiment, the inhibitor inhibits one or more types of scavenger receptors. In one embodiment, the inhibitor specifically inhibits a particular type of scavenger receptor. In one embodiment, the composition comprises one or more inhibitors, wherein each inhibitor inhibits one or more types of scavenger receptors.

In one embodiment, the composition comprises an inhibitor of scavenger receptor B1. In one embodiment, the composition comprises an inhibitor of the expression of one or more scavenger receptors. For example, in one embodiment, the composition comprises an isolated nucleic acid (e.g., siRNA, ribozyme, antisense RNA, etc.) that reduces the expression level of one or more scavenger receptors in a cell. In one embodiment, the composition comprises an inhibitor of the activity of one or more scavenger receptors. For example, in one embodiment, the composition comprises a nucleic acid, peptide, antibody, small molecule, antagonist, aptamer, or peptidomimetic that reduces the activity of one or more scavenger receptors. In one embodiment, the composition comprises the peptide inhibitor D-4F. In one embodiment, the composition comprises an isolated nucleic acid encoding D-4F. In one embodiment, the composition comprises the small molecule inhibitor BLT-1. In one embodiment, the composition comprises the small molecule inhibitor ITX-5061

In one embodiment, the composition comprises clodronate. For example, in one embodiment, the composition comprises a combination of an inhibitor of one or more scavenger receptors and clodronate.

In one embodiment, the present invention includes a method for increasing frequency, amount, or presence of RBCs in peripheral blood. In one embodiment, the method is used to treat certain forms of anemia, including for example sickle cell anemia. In one embodiment, the method is used to treat any cholesterol abnormality associated with red blood cell deformation. In one embodiment, the method comprises administering to a subject in need an effective amount of a composition comprising an inhibitor of one or more scavenger receptors. In one embodiment, the method comprises administering to a subject in need a composition comprising an inhibitor of scavenger receptor B1. In one embodiment, the method comprises administering to a subject in need a composition comprising clodronate. In one embodiment, the method comprises administering to a subject in need a composition comprising clodronate and an inhibitor of one or more scavenger receptors.

In one embodiment, the present invention includes a non-human animal in which the activity, expression, or both of one or more scavenger receptors are inhibited. In certain instances the inhibition of the one or more scavenger receptors allows for increased human RBCs in the periphery following engraftment of the animal with human cells. Inhibition of one or more scavenger receptors allows for production of a humanized mouse model where human erythroid cells can mature and remain sustained within the circulatory system. In one embodiment, the animal is treated with an inhibitor of one or more scavenger receptors. In one embodiment, the animal is genetically modified to reduce or prevent expression of one or more scavenger receptors. In one aspect, the genetically modified non-human animal expresses at least one of human EPO, human M-CSF, human IL-3/GM-CSF, human SIRPα, and human TPO. The invention also relates to methods of generating and methods of using the genetically modified non-human animals described herein. In some embodiments, the genetically modified non-human animal is a mouse. In some embodiments, the genetically modified non-human animal is an immunodeficient mouse. In a particular embodiment, the immunodeficient mouse is a RAG2^{-/-} γ_{c}^{-/-} mouse. In another particular embodiment, the genetically modified non-human animal of the invention expresses human EPO and at least one of human M-CSF, human IL-3/GM-CSF, and human TPO, and does not express RAG2 or γ_{c}. In another particular embodiment, the genetically modified non-human animal of the invention expresses human EPO, and at least one of human M-CSF, human IL-3/GM-CSF, human SIRPα, and human TPO, and does not express RAG2 or γ_{c} (referred to herein as MISTERG). In some embodiments, the genetically modified non-human animals described herein are engrafted with a human hematopoietic cell.

In various embodiments, the human hematopoietic cell engrafted, genetically modified non-human animals of the invention are useful for the in vivo evaluation of the growth and differentiation of hematopoietic and immune cells, for the in vivo evaluation of human hematopoiesis, for the in vivo evaluation of erythropoiesis, for the in vivo evaluation of cancer cells, for the in vivo assessment of an immune response, for the in vivo evaluation of vaccines and vaccination regimens, for the use in testing the effect of agents that modulate cancer cell growth or survival, for the in vivo evaluation of a treatment of cancer, for the in vivo evaluation of malaria infection, for the in vivo evaluation of a treatment of malaria infection, for the in vivo production and collection of immune mediators, including human antibodies, and for use in testing the effect of agents that modulate hematopoietic and immune cell function.

### Inhibitors

In one embodiment, the present invention provides a composition for increasing the frequency, amount, or presence of RBCs in peripheral blood. In certain embodiments, the composition inhibits the expression, activity, or both of one or more scavenger receptors. In one embodiment, the composition inhibits the expression, activity, or both, of scavenger receptor B1.

An inhibitor of one or more scavenger receptors is any compound, molecule, or agent that reduces, inhibits, or prevents the function of one or more scavenger receptors. For example, an inhibitor of one or more scavenger receptors is any compound, molecule, or agent that reduces expression, activity, or both of the one or more scavenger receptors. In one embodiment, an inhibitor of one or more scavenger receptors a nucleic acid, a peptide, a small molecule, a siRNA, a ribozyme, an antisense, an antagonist, an aptamer, a peptidomimetic, or any combination thereof.

### Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

Small molecule inhibitors of scavenger receptors include, but are not limited to BLT-1 (2-Hexyl-1-cyclopentanone thiosemicarbazone (C₁₂H₂₃N₃S)) and ITX-5061 (iTherx). BLT-1 and ITX-5061 are both inhibitors of scavenger receptor B1. BLT-1 is shown herein to increase the amount of human RBCs in peripheral blood.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Peptide Inhibitors

In one embodiment the invention includes an isolated peptide inhibitor that inhibits one or more scavenger receptors. For example, in one embodiment, the peptide inhibitor of the invention inhibits the one or more scavenger receptors directly by binding to the one or more scavenger receptors thereby preventing the normal functional activity of the one or more scavenger receptors. In another embodiment, the peptide inhibitor of the invention inhibits the one or more scavenger receptors by competing with endogenous scavenger receptor. In yet another embodiment, the peptide inhibitor of the invention inhibits the activity of the one or more scavenger receptors by acting as a transdominant negative mutant.

In one embodiment, the inhibitor of one or more scavenger receptors is an isolated peptide comprising the amino acid sequence DWLKAFYDKVAEKLKEAF (SEQ ID NO: 1). In one embodiment, the inhibitor of the one or more scavenger receptors is D-4F (Ac-D-W-L-K-A-F-Y-D-K-V-A-E-K-L-K-E-A-F—NH₂, SEQ ID NO: 2). D-4F is an apolipoprotein A-I mimetic peptide, shown herein to increase the amount of peripheral RBCs.

In one embodiment, the invention includes an isolated peptide inhibitor that inhibits scavenger receptor B1. For example, in one embodiment, the peptide inhibitor of the invention inhibits scavenger receptor B1 directly by binding to scavenger receptor B1 thereby preventing the normal functional activity of scavenger receptor B1. In another embodiment, the peptide inhibitor of the invention inhibits scavenger receptor B1 by competing with endogenous scavenger receptor B1. In yet another embodiment, the peptide inhibitor of the invention inhibits the activity of scavenger receptor B1 by acting as a transdominant negative mutant.

In one embodiment, the invention includes variants of the peptides of the invention. In one embodiment, variants differ from naturally-occurring peptides by conservative amino acid sequence differences or by modifications which do not affect sequence, or by both. For example, conservative amino acid changes may be made, which although they alter the primary sequence of the peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

In one embodiment, the peptide of the invention comprises a peptide having at least 75% homology, at least 80% homology, at least 85% homology, at least 90% homology, at least 95% homology, or at least 99% homology with the D-4F peptide inhibitor. In a further embodiment, the peptide of the invention comprise D-, L-, and unnatural isomers of amino acids.

As known in the art the “similarity” between two peptides is determined by comparing the amino acid sequence and its conserved amino acid substitutes of one polypeptide to a sequence of a second peptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)).

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12: 429-456) excellent solubility in aqueous solution, low immunogenicity and antigenicity (Dreborg et al., 1990, Crit. Rev. Ther. Drug Carrier Syst. 6: 315-365). PEG-conjugated or “PEGylated” protein therapeutics, containing single or multiple chains of polyethylene glycol on the protein, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters. Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

Biological preparation of a peptide of the invention involves expression of a nucleic acid encoding a desired peptide. An expression cassette comprising such a coding sequence may be used to produce a desired peptide for use in the method of the invention.

Examples of biological methods to prepare the peptides of the present invention may utilize methods provided in published US Patent Application Number US 2009/0069241, which is incorporated herein in its entirety.

To ensure that the polypeptide obtained from either chemical or biological synthetic techniques is the desired polypeptide, analysis of the peptide composition can be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

### Antibody Inhibitors

The invention also contemplates an inhibitor of one or more scavenger receptors comprising an antibody, or antibody fragment, specific for one or more scavenger receptors. That is, the antibody can inhibit one or more scavenger receptors to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain F_{V} molecule, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Nucleic Acid Inhibitors

In one embodiment, the invention includes an isolated nucleic acid. For example, in one embodiment, the composition comprises a nucleic acid sequence that encodes the peptide inhibitor of the invention. In one embodiment, the invention includes nucleic acid sequences corresponding to the amino acid sequences of D-4F.

In some instances the inhibitor is an siRNA or antisense molecule, which inhibits one or more scavenger receptors. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (Current Protocols in Molecular Biology, John Wiley & Sons, New York) and as described elsewhere herein.

In another aspect of the invention, one or more scavenger receptors can be inhibited by way of inactivating and/or sequestering the one or more scavenger receptors. As such, inhibiting the activity of one or more scavenger receptors can be accomplished by using a transdominant negative mutant.

In one embodiment, siRNA is used to decrease the level of one or more scavenger receptors protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of one or more scavenger receptors using RNAi technology.

In another aspect, the invention includes a vector comprising an siRNA or antisense polynucleotide. Preferably, the siRNA or antisense polynucleotide is capable of inhibiting the expression of a target polypeptide, wherein the target polypeptide is one or more scavenger receptors. The incorporation of a desired polynucleotide into a vector and the choice of vectors is well-known in the art as described in, for example, Sambrook et al. (2012, Molecular Cloning: A Laboratory Manual, Cold Spring Harbor Laboratory, New York), and in Ausubel et al. (2005, Current Protocols in Molecular Biology, John Wiley & Sons, New York), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in Ausubel et al. (2005, Current Protocols in Molecular Biology, John Wiley & Sons, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art. In a particular embodiment, the vector is a vector useful for transforming animal cells.

In one embodiment, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012, Molecular Cloning: A Laboratory Manual, Cold Spring Harbor Laboratory, New York). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In one embodiment of the invention, an antisense nucleic acid sequence which is expressed by a plasmid vector is used to inhibit the protein expression of one or more scavenger receptors. The antisense expressing vector is used to transfect a mammalian cell or the mammal itself, thereby causing reduced endogenous expression of one or more scavenger receptors.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In the context of an expression vector, the vector can be readily introduced into a host cell, e.g., mammalian, bacterial, yeast or insect cell by any method in the art. Coding sequences for a desired peptide of the invention may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

The expression vector can be transferred into a host cell by physical, biological or chemical means. Physical methods for introducing a polynucleotide into a host cell include calcium phosphate precipitation, lipofection, particle bombardment, microinjection, electroporation, photoporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (2005, Current Protocols in Molecular Biology, John Wiley & Sons, New York). Thus, the invention encompasses expression vectors encoding a PEDF peptide or fusion protein of the invention, as well as cells comprising such vectors.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from baculovirus, papovavirus, vaccinia virus, pseudorabies virus, fowl pox virus, lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art. In the case where a non-viral delivery system is utilized, a preferred delivery vehicle is a liposome. The above-mentioned delivery systems and protocols can be found in Gene Targeting Protocols, 2.sup.nd ed., Kmiec ed., Humana Press, Totowa, N.J., pp 1-35 (2002) and Gene Transfer and Expression Protocols, Vol. 7, (Methods in Molecular Biology) Murray ed., Humana Press, Totowa, N.J., pp 81-89 (1991).

In one embodiment of the invention, a ribozyme is used to inhibit protein expression of one or more scavenger receptors. Ribozymes useful for inhibiting the expression of a target molecule may be designed by incorporating target sequences into the basic ribozyme structure which are complementary, for example, to the mRNA sequence encoding one or more scavenger receptors. Ribozymes targeting one or more scavenger receptors may be synthesized using commercially available reagents (Applied Biosystems, Inc., Foster City, Calif.) or they may be genetically expressed from DNA encoding them.

### Treatment Methods

The present invention provides methods of increasing the number or percentage of RBCs in the peripheral blood of a subject in need thereof. In some embodiments, the present invention provides methods of treating or preventing anemia in a subject in need thereof. In one embodiment, the method comprises administering an effective amount of a composition which increases the frequency, amount or presence of RBCs in peripheral blood. In certain embodiments, the method of the invention comprises administering to a subject an effective amount of a composition that inhibits the expression, activity, or both of one or more scavenger receptors. For example, in one embodiment, the method of the invention comprises administering to a subject an effective amount of a composition that inhibits the expression, activity, or both of scavenger receptor B1. In another embodiment, the method of the invention comprises administering to a subject an effective amount of a composition comprising clodronate and an inhibitor of the expression, activity, or both of one or more scavenger receptors. In one embodiment, the method of the invention comprises administering to a subject an effective amount of a first composition comprising clodronate and an effective amount of a second composition comprising an inhibitor of the expression, activity, or both of one or more scavenger receptors.

In one embodiment, the method comprises administering to a subject an effective amount of a composition comprising D-4F. In one embodiment, the method comprises administering to a subject an effective amount of a composition comprising an isolated nucleic acid sequence encoding D-4F, or functional equivalent thereof. In one embodiment, the method comprises administering to a subject an effective amount of a composition comprising BLT-1. In one embodiment, the method comprises administering to a subject an effective amount of a composition comprising ITX-5061

The invention includes methods for the treatment of any disease, disorder, or condition which is caused or characterized by reduced or diminished levels of peripheral RBCs. For example, in one embodiment, the invention includes a method for the treatment of anemia, including for example, sickle cell anemia. In one embodiment, the invention provides a method for the treatment of a cholesterol abnormality associated with red blood cell deformation.

The activity of one or more scavenger receptors can be inhibited using any method known to the skilled artisan. Examples of methods that inhibit the activity of one or more scavenger receptors, include but are not limited to, inhibiting expression of an endogenous gene encoding the one or more scavenger receptors, decreasing expression of mRNA encoding the one or more scavenger receptors, and inhibiting the function, activity, or stability of the one or more scavenger receptors. An inhibitor of the one or more scavenger receptors may therefore be a compound that decreases expression of a gene encoding the one or more scavenger receptors, decreases RNA half-life, stability, or expression of a mRNA encoding the one or more scavenger receptors, or inhibits function, activity or stability of the one or more scavenger receptors. An inhibitor of the one or more scavenger receptors may be any type of compound, including but not limited to, a peptide, a nucleic acid, an aptamer, a peptidometic, and a small molecule, or combinations thereof.

Inhibition of the one or more scavenger receptors may be accomplished either directly or indirectly. For example the one or more scavenger receptors may be directly inhibited by compounds or compositions that directly interact with the one or more scavenger receptors, such as antibodies. Alternatively, the one or more scavenger receptors may be inhibited indirectly by compounds or compositions that inhibit downstream effectors of the one or more scavenger receptors, or upstream regulators which up-regulate expression of the one or more scavenger receptors.

Decreasing expression of an endogenous gene includes providing a specific inhibitor of gene expression. Decreasing expression of mRNA or protein includes decreasing the half-life or stability of mRNA or decreasing expression of mRNA. Methods of decreasing expression of the one or more scavenger receptors include, but are not limited to, methods that use an siRNA, a microRNA, an antisense nucleic acid, a ribozyme, an expression vector encoding a transdominant negative mutant, a peptide, a small molecule, and combinations thereof.

Administration of an inhibitor of the one or more scavenger receptors in a method of treatment can be achieved in a number of different ways, using methods known in the art.

It will be appreciated that an inhibitor of the one or more scavenger receptors of the invention may be administered to a subject either alone, or in conjunction with another therapeutic agent.

In one embodiment, the inhibitor is administered to a subject. The inhibitor may also be a hybrid or fusion composition to facilitate, for instance, delivery to target cells or efficacy. In one embodiment, a hybrid composition may comprise a tissue-specific targeting sequence.

The therapeutic and prophylactic methods of the invention thus encompass the use of pharmaceutical compositions comprising an inhibitor of one or more scavenger receptors to practice the methods of the invention. The pharmaceutical compositions useful for practicing the invention may be administered to deliver a dose of from ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

### Genetically Modified Non-Human Animals

The invention includes a genetically modified non-human animal, wherein the non-human animal is modified to not express one or more scavenger receptors, or is modified to exhibit reduced expression of one or more scavenger receptors. For example, in one embodiment, the non-human animal is genetically modified such that animal comprises only one functional copy of the gene encoding scavenger receptor B1 (i.e., heterozygous), referred to herein as SRB1^{+/-}. In one embodiment, the non-human animal is genetically modified such that both copies of the gene encoding scavenger receptor B1 are absent or otherwise defective, referred to herein as SRB1^{-/-}.

In one embodiment, the genetically modified non-human animal does not express scavenger receptor B1, or exhibits reduced scavenger receptor B1 expression, and further expresses at least one of human EPO, human M-CSF, human IL-3/GM-CSF, human SIRPα, human TPO, and any combination thereof. In one particular embodiment, the genetically modified non-human animal of the invention expresses human M-CSF, human IL-3/GM-CSF, human TPO, and human EPO and does not express RAG2 or γ_{c}. In another particular embodiment, the genetically modified non-human animal of the invention expresses human M-CSF, human IL-3/GM-CSF, human SIRPα, human TPO, and human EPO and does not express RAG2 or γ_{c}. In another particular embodiment, the genetically modified non-human animal of the invention expresses human M-CSF, human IL-3/GM-CSF, human SIRPα, and human TPO, and does not express RAG2 or γ_{c}. In another particular embodiment, the genetically modified non-human animal of the invention expresses human IL-3/GM-CSF, human SIRPα, human TPO, and human EPO and does not express RAG2 or γ_{c}.

In one aspect, the present invention provides a genetically modified non-human animal that expresses at least one of human EPO, human M-CSF, human IL-3/GM-CSF, human SIRPα, human TPO, and any combination thereof; and is treated with a composition, or is genetically modified, to enhance or increase human RBCs in peripheral blood. For example, in one embodiment, the genetically modified non-human animal is treated with a composition that inhibits the activity, expression, or both of one or more scavenger receptors. For example, in one embodiment, the genetically modified non-human animal is treated with a composition that inhibits the activity, expression, or both of scavenger receptor B1. As further described elsewhere herein, in certain embodiments, the inhibitor is a nucleic acid, a peptide, a small molecule, a siRNA, a ribozyme, an antisense nucleic acid, an antagonist, an aptamer, a peptidomimetic, or any combination thereof. In one embodiment, the genetically modified non-human animal is genetically modified to not express one or more scavenger receptors. For example, in one embodiment, the genetically modified non-human animal is genetically modified to not express scavenger receptor B1. In one embodiment, the genetically modified non-human animal is treated with clodronate. For example, in one embodiment, the genetically modified non-human animal is treated with a composition comprising a combination of clodronate and an inhibitor of one or more scavenger receptors. In one embodiment, the genetically modified non-human animal modified to not express one or more scavenger receptors is treated with a composition comprising clodronate. In one particular embodiment, the genetically modified non-human animal of the invention expresses human M-CSF, human IL-3/GM-CSF, human TPO, and human EPO and does not express RAG2 or γ_{c}. In another particular embodiment, the genetically modified non-human animal of the invention expresses human M-CSF, human IL-3/GM-CSF, human SIRPα, human TPO, and human EPO and does not express RAG2 or γ_{c}. In another particular embodiment, the genetically modified non-human animal of the invention expresses human IL-3/GM-CSF, human SIRPα, human TPO, and human EPO and does not express RAG2 or γ_{c}.

In some embodiments, the genetically modified non-human animal that expresses a human nucleic acid also expresses the corresponding non-human animal nucleic acid. In other embodiments, the genetically modified non-human animal that expresses a human nucleic acid does not also express the corresponding non-human animal nucleic acid. In some embodiments, the genetically modified animal is an animal having one or more genes knocked out to render the animal an immunodeficient animal, as elsewhere described herein. To create a genetically modified non-human animal, a nucleic acid encoding a human protein can be incorporated into a recombinant expression vector in a form suitable for expression of the human protein in a non-human host cell. In various embodiments, the recombinant expression vector includes one or more regulatory sequences operatively linked to the nucleic acid encoding the human protein in a manner which allows for transcription of the nucleic acid into mRNA and translation of the mRNA into the human protein. The term “regulatory sequence” is art-recognized and intended to include promoters, enhancers and other expression control elements (e.g., polyadenylation signals). Such regulatory sequences are known to those skilled in the art and are described in 1990, Goeddel, Gene Expression Technology: Methods in Enzymology 185, Academic Press, San Diego, Calif. It should be understood that the design of the expression vector may depend on such factors as the choice of the host cell to be transfected and/or the amount of human protein to be expressed.

A genetically modified animal can be created, for example, by introducing a nucleic acid encoding the human protein (typically linked to appropriate regulatory elements, such as a constitutive or tissue-specific enhancer) into an oocyte, e.g., by microinjection, and allowing the oocyte to develop in a female foster animal. Intronic sequences and polyadenylation signals can also be included in the transgene to increase the efficiency of expression of the transgene. Methods for generating genetically modified animals, particularly animals such as mice, have become conventional in the art and are described, for example, in U.S. Pat. Nos. 4,736,866 and 4,870,009 and 1986, Hogan et al., A Laboratory Manual, Cold Spring Harbor, N.Y., Cold Spring Harbor Laboratory. A genetically modified founder animal can be used to breed additional animals carrying the transgene. Genetically modified animals carrying a transgene encoding the human protein of the invention can further be bred to other genetically modified animals carrying other transgenes, or be bred to knockout animals, e.g., a knockout animal that does not express one or more of its genes. In various embodiments, the genetically modified animal of the invention is a mouse, a rat or a rabbit.

In some embodiments, the genetically modified animal of the invention expresses one or more human nucleic acids from the non-human animal's native promoter and native regulatory elements. In other embodiments, the genetically modified animal of the invention expresses a human nucleic acid from the native human promoter and native regulatory elements. The skilled artisan will understand that the genetically modified animal of the invention includes genetically modified animals that express at least one human nucleic acid from any promoter. Examples of promoters useful in the invention include, but are not limited to, DNA pol II promoter, PGK promoter, ubiquitin promoter, albumin promoter, globin promoter, ovalbumin promoter, SV40 early promoter, the Rous sarcoma virus (RSV) promoter, retroviral LTR and lentiviral LTR. Promoter and enhancer expression systems useful in the invention also include inducible and/or tissue-specific expression systems.

In some embodiments, the invention includes genetically modified immunodeficient animals having a genome that includes a nucleic acid encoding a human polypeptide operably linked to a promoter, wherein the animal expresses the encoded human polypeptide. In various embodiments, the invention includes genetically modified immunodeficient non-human animals having a genome that comprises an expression cassette that includes a nucleic acid encoding at least one human polypeptide, wherein the nucleic acid is operably linked to a promoter and a polyadenylation signal and further contains an intron, and wherein the animal expresses the encoded human polypeptide.

In various embodiments, various methods are used to introduce a human nucleic acid sequence into an immunodeficient animal to produce a genetically modified immunodeficient animal that expresses a human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006, Genetically Engineered Mice Handbook, CRC Press), Hofker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol Biol., Humana Press), Meyer et al. (2010, Proc. Nat. Acad. Sci. USA 107:15022-15026), and Gibson (2004, A Primer Of Genome Science 2^{nd} ed. Sunderland, Mass.: Sinauer), U.S. Pat. No. 6,586,251, Rathinam et al. (2011, Blood 118:3119-28), Willinger et al., (2011, Proc Natl Acad Sci USA, 108:2390-2395), Rongvaux et al., (2011, Proc Natl Acad Sci USA, 108:2378-83) and Valenzuela et al. (2003, Nat Biot 21:652-659).

In some embodiments, the compositions and methods of the invention comprise genetically modified immunodeficient animals deficient in B cell and/or T cell number and/or function, alone, or in combination with a deficiency in NK cell number and/or function (for example, due to an IL2 receptor gamma chain deficiency (i.e., γ_{c}^{-/-})), and having a genome that comprises a human nucleic acid operably linked to a promoter, wherein the animal expresses the encoded human polypeptide. The generation of the genetically modified animal of the invention can be achieved by methods such as DNA injection of an expression construct into a preimplantation embryo or by use of stem cells, such as embryonic stem (ES) cells or induced pluripotent stem (iPS) cells.

In one embodiment, the human nucleic acid is expressed by the native regulatory elements of the human gene. In other embodiments, the human nucleic acid is expressed by the native regulatory elements of the non-human animal. In other embodiments, human nucleic acid is expressed from a ubiquitous promoter. Nonlimiting examples of ubiquitous promoters useful in the expression construct of the compositions and methods of the invention include, a 3-phosphoglycerate kinase (PGK-1) promoter, a beta-actin promoter, a ROSA26 promoter, a heat shock protein 70 (Hsp70) promoter, an EF-1 alpha gene encoding elongation factor 1 alpha (EF1) promoter, an eukaryotic initiation factor 4A (eIF-4A1) promoter, a chloramphenicol acetyltransferase (CAT) promoter and a CMV (cytomegalovirus) promoter.

In other embodiments, the human nucleic acid is expressed from a tissue-specific promoter. Non-limiting examples of tissue-specific promoters useful in the expression construct of the compositions and methods of the invention include a promoter of a gene expressed in the hematopoietic system, such as a M-CSF promoter, an IL-3 promoter, a GM-CSF promoter, a SIRPα promoter, a TPO promoter, a EPO promoter, an IFN-β promoter, a Wiskott-Aldrich syndrome protein (WASP) promoter, a CD45 (also called leukocyte common antigen) promoter, a Flt-1 promoter, an endoglin (CD105) promoter and an ICAM-2 (Intracellular Adhesion Molecule 2) promoter. These and other promoters useful in the compositions and methods of the invention are known in the art as exemplified in Abboud et al. (2003, J. Histochem & Cytochem. 51:941-949), Schorpp et al. (1996, NAR 24:1787-1788), McBurney et al. (1994, Devel. Dynamics, 200:278-293) and Majumder et al. (1996, Blood 87:3203-3211). Further to comprising a promoter, one or more additional regulatory elements, such as an enhancer element or intron sequence, is included in various embodiments of the invention. Examples of enhancers useful in the compositions and methods of the invention include, but are not limited to, a cytomegalovirus (CMV) early enhancer element and an SV40 enhancer element. Examples of intron sequences useful in the compositions and methods of the invention include, but are not limited to, the beta globin intron or a generic intron. Other additional regulatory elements useful in some embodiments of the invention include, but are not limited to, a transcription termination sequence and an mRNA polyadenylation (pA) sequence.

In some embodiments, the methods of introduction of the human nucleic acid expression construct into a preimplantation embryo include linearization of the expression construct before it is injected into a preimplantation embryo. In preferred embodiments, the expression construct is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

In other embodiments, the expression construct may be transfected into stem cells (ES cells or iPS cells) using well-known methods, such as electroporation, calcium-phosphate precipitation and lipofection. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be microinjected into preimplantation embryos. For a detailed description of methods known in the art useful for the compositions and methods of the invention, see Nagy et al., (2002, Manipulating the Mouse Embryo: A Laboratory Manual, 3rd edition, Cold Spring Harbor Laboratory Press), Nagy et al. (1990, Development 110:815-821), U.S. Pat. No. 7,576,259, U.S. Pat. No. 7,659,442, U.S. Pat. No. 7,294,754, and Kraus et al. (2010, Genesis 48:394-399).

The genetically modified non-human animals of the invention can be crossed to immunodeficient animal to create an immunodeficient animal expressing at least one human nucleic acid. Various embodiments of the invention provide genetically modified animals that include a human nucleic acid in substantially all of their cells, as well as genetically modified animals that include a human nucleic acid in some, but not all their cells. One or multiple copies, adjacent or distant to one another, of the human nucleic acid may be integrated into the genome of the cells of the genetically modified animals.

In some embodiments, the invention is a genetically modified non-human mouse engrafted with at least one human hematopoietic cell. In other embodiments, the invention is a method of engrafting human hematopoietic cells in a genetically modified non-human animal. The engrafted human hematopoietic cells useful in the compositions and methods of the invention include any human hematopoietic cell. Non-limiting examples of human hematopoietic cells useful in the invention include, but are not limited to, HSC, HSPC, leukemia initiating cells (LIC), RBC, and hematopoietic cells of any lineage at any stage of differentiation, including terminally differentiated hematopoietic cells of any lineage. Such hematopoietic cells can be derived from any tissue or location of a human donor, including, but not limited to, bone marrow, peripheral blood, liver, fetal liver, or umbilical cord blood. Such hematopoietic cells can be isolated from any human donor, including healthy donors, as well as donors with disease, such as cancer, including leukemia.

In other embodiments, the invention is a method of engrafting human hematopoietic cells in a genetically modified non-human animal. In some embodiments, the genetically modified non-human animal into which human hematopoietic cells are engrafted is an immunodeficient animal. Engraftment of hematopoietic cells in the genetically modified animal of the invention is characterized by the presence of human hematopoietic cells in the engrafted animal. In particular embodiments, engraftment of hematopoietic cells in an immunodeficient animal is characterized by the presence of differentiated human hematopoietic cells in the engrafted animal in which hematopoietic cells are provided, as compared with appropriate control animals.

In some embodiments, the animals of the invention are transplanted with human cancer cells (e.g., human solid tumors, etc.) in addition to human hematopoietic cells. In various embodiments, the human cancer cells can be a cancer cell line or primary human cancer cell isolated from a patient, from any of many different types of cancer (including, by way of non-limiting examples, melanoma, breast cancer, lung cancer, etc.) In some embodiments, the human cancer cell and the HSPC are isolated from the same patient and transplanted into the same non-human animal.

The genetically modified non-human animals provided in various embodiments of the present invention have various utilities such as, but not limited to, for use as models of growth and differentiation of hematopoietic and immune cells, for the in vivo evaluation of human hematopoiesis, for the in vivo evaluation of erythropoiesis, for the in vivo evaluation of cancer cells, for the in vivo assessment of an immune response, for the in vivo evaluation of vaccines and vaccination regimens, for the use in testing the effect of agents that modulate cancer cell growth or survival, for the in vivo evaluation of a treatment of cancer, for the in vivo evaluation of malaria infection, for the in vivo evaluation of a treatment of malaria infection, for the in vivo production and collection of immune mediators, including human antibodies, and for use in testing the effect of agents that modulate hematopoietic and immune cell function.

Engraftment of human hematopoietic cells in genetically modified and/or immunodeficient non-human animals has traditionally required conditioning prior to administration of the hematopoietic cells, either sub-lethal irradiation of the recipient animal with high frequency electromagnetic radiation, generally using gamma or X-ray radiation, or treatment with a radiomimetic drug such as busulfan or nitrogen mustard. Conditioning is believed to reduce numbers of host hematopoietic cells, create appropriate microenvironmental factors for engraftment of human hematopoietic cells, and/or create microenvironmental niches for engraftment of human hematopoietic cells. Standard methods for conditioning are known in the art, such as described herein and in Hayakawa et al, 2009, Stem Cells, 27(1):175-182. Methods for engraftment of human hematopoietic cells in immunodeficient animals are provided according to embodiments of the present invention which include providing human hematopoietic cells to the immunodeficient animals, with or without irradiating the animals prior to administration of the hematopoietic cells. Methods for engraftment of human hematopoietic cells in immunodeficient animals are provided according to embodiments of the present invention which include providing human hematopoietic cells to the genetically modified non-human animals of the invention, with or without, administering a radiomimetic drug, such as busulfan or nitrogen mustard, to the animals prior to administration of the hematopoietic cells.

In some embodiments, the methods of hematopoietic cell engraftment in a genetically modified non-human animal according to embodiments of the present invention include providing human hematopoietic cells to a genetically modified animal of the invention as elsewhere described here. In some embodiments, the genetically modified non-human animal of the invention is an immunodeficient animal that is deficient in non-human B cell number and/or function, non-human T cell number and/or function, and/or non-human NK cell number and/or function. In other embodiments, the immunodeficient animal has severe combined immune deficiency (SCID). SCID refers to a condition characterized by the absence of T cells and lack of B cell function. Examples of SCID include: X-linked SCID, which is characterized by gamma chain gene mutations in the IL2RG gene and the lymphocyte phenotype T(−) B(+) NK(−); and autosomal recessive SCID characterized by Jak3 gene mutations and the lymphocyte phenotype T(−) B(+) NK(−), ADA gene mutations and the lymphocyte phenotype T(−) B(−) NK(−), IL-7R alpha-chain mutations and the lymphocyte phenotype T(−) B(+) NK(+), CD3 delta or epsilon mutations and the lymphocyte phenotype T(−) B(+) NK(+), RAG1/RAG2 mutations and the lymphocyte phenotype T(−) B(−) NK(+), Artemis gene mutations and the lymphocyte phenotype T(−) B(−) NK(+), CD45 gene mutations and the lymphocyte phenotype T(−) B(+) NK(+). In some embodiments, the genetically modified non-human animal of the invention is RAG1^{-/-}.

In some embodiments, the methods of hematopoietic cell engraftment in a genetically modified animal according to embodiments of the present invention include providing a human hematopoietic cell to in a genetically modified non-human animal having the severe combined immunodeficiency mutation (Prkdc^{scid}), commonly referred to as the scid mutation. The scid mutation is well-known and located on mouse chromosome 16 as described in Bosma et al. (1989, Immunogenetics 29:54-56). Mice homozygous for the scid mutation are characterized by an absence of functional T cells and B cells, lymphopenia, hypoglobulinemia and a normal hematopoietic microenvironment. The scid mutation can be detected, for example, by detection of markers of the scid mutation using well-known methods.

In other embodiments, the methods of hematopoietic cell engraftment in a genetically modified animal according to embodiments of the present invention include providing human hematopoietic cells to genetically modified immunodeficient non-human animal having an IL2 receptor gamma chain deficiency, either alone, or in combination with, the severe combined immunodeficiency (scid) mutation. The term “IL2 receptor gamma chain deficiency” refers to decreased IL2 receptor gamma chain. Decreased IL2 receptor gamma chain can be due to gene deletion or mutation. Decreased IL2 receptor gamma chain can be detected, for example, by detection of IL2 receptor gamma chain gene deletion or mutation and/or detection of decreased IL2 receptor gamma chain expression using well-known methods.

In addition to the naturally occurring human nucleic acid and amino acid sequences, the term encompasses variants of human nucleic acid and amino acid sequences. As used herein, the term “variant” defines either an isolated naturally occurring genetic mutant of a human or a recombinantly prepared variation of a human, each of which contain one or more mutations compared with the corresponding wild-type human. For example, such mutations can be one or more amino acid substitutions, additions, and/or deletions. The term “variant” also includes non-human orthologues. In some embodiments, a variant polypeptide of the present invention has at least 90%, 91%, 92%, 93%, 94%, 95%, 96%, 97%, 98% or 99% identity to a wild-type human polypeptide.

The percent identity between two sequences is determined using techniques as those described elsewhere herein. Mutations can be introduced using standard molecular biology techniques, such as site-directed mutagenesis and PCR-mediated mutagenesis. One of skill in the art will recognize that one or more amino acid mutations can be introduced without altering the functional properties of human proteins.

Conservative amino acid substitutions can be made in human proteins to produce human protein variants. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

Human variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, methylhistidine, and ornithine.

Human variants are encoded by nucleic acids having a high degree of identity with a nucleic acid encoding a wild-type human protein. The complement of a nucleic acid encoding a human variant protein specifically hybridizes with a nucleic acid encoding a wild-type human protein under high stringency conditions.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

Nucleic acids encoding a human variant can be isolated or generated recombinantly or synthetically using well-known methodology.

Isolation of human hematopoietic cells, administration of the human hematopoietic cells to a host animal and methods for assessing engraftment thereof are well-known in the art. Hematopoietic cells for administration to host animal can be obtained from any tissue containing hematopoietic cells such as, but not limited to, umbilical cord blood, bone marrow, peripheral blood, cytokine or chemotherapy-mobilized peripheral blood and fetal liver. Hematopoietic cells can be administered into newborn or adult animals by administration via various routes, such as, but not limited to, intravenous, intrahepatic, intraperitoneal, intrafemoral and/or intratibial.

Engraftment of human hematopoietic cells in the genetically modified animal of the invention can be assessed by any of various methods, such as, but not limited to, flow cytometric analysis of cells in the animals to which the human hematopoietic cells are administered at one or more time points following the administration of hematopoietic cells.

Exemplary methods of isolating human hematopoietic cells, of administering human hematopoietic cells to a host animal, and of assessing engraftment of the human hematopoietic cells in the host animal are described herein and in Pearson et al. (2008, Curr. Protoc. Immunol. 81:1-15), Ito et al. (2002, Blood 100:3175-3182), Traggiai et al. (2004, Science 304:104-107), Ishikawa et al. (2005, Blood 106:1565-1573), Shultz et al. (2005, J. Immunol. 174:6477-6489) and Holyoake et al. (1999, Exp Hematol. 27:1418-27).

In some embodiments of the invention, the human hematopoietic cells are isolated from an original source material to obtain a population of cells enriched for a particular hematopoietic cell population (e.g., HSCs, HSPCs, LICs, CD34+, CD34−, lineage specific marker, etc.). The isolated hematopoietic cells may or may not be a pure population. In one embodiment, hematopoietic cells useful in the compositions and methods of the invention are depleted of cells having a particular marker, such as CD34. In another embodiment, hematopoietic cells useful in the compositions and methods of the invention are enriched by selection for a marker, such as CD34. In some embodiments, hematopoietic cells useful in the compositions and methods of the invention are a population of cells in which CD34+ cells constitute about 1-100% of the cells, although in certain embodiments, a population of cells in which CD34+ cells constitute fewer than 1% of total cells can also be used. In certain embodiments, the hematopoietic cells useful in the compositions and methods of the invention are a T cell-depleted population of cells in which CD34+ cells make up about 1-3% of total cells, a lineage-depleted population of cells in which CD34+ cells make up about 50% of total cells, or a CD34+ positive selected population of cells in which CD34+ cells make up about 90% of total cells.

The number of hematopoietic cells administered is not considered limiting with regard to the generation of a human hematopoietic and/or immune system in a genetically modified non-human animal expressing at least one human gene. Thus, by way of non-limiting example, the number of hematopoietic cells administered can range from about 1×10³ to about 1×10⁷, although in various embodiments, more or fewer can also be used. By way of another non-limiting example, the number of HSPCs administered can range from about 3×10³ to about 1×10⁶ CD34+ cells when the recipient is a mouse, although in various embodiments, more or fewer can also be used. For other species of recipient, the number of cells that need to be administered can be determined using only routine experimentation.

Generally, engraftment can be considered successful when the number (or percentage) of human hematopoietic cells present in the genetically modified non-human animal is greater than the number (or percentage) of human cells that were administered to the non-human animal, at a point in time beyond the lifespan of the administered human hematopoietic cells. Detection of the progeny of the administered hematopoietic cells can be achieved by detection of human DNA in the recipient animal, for example, or by detection of intact human hematopoietic cells, such as by the detection of the human cell surface marker, such as CD45 for example. Serial transfer of human hematopoietic cells from a first recipient into a secondary recipient, and engraftment of human hematopoietic cells in the second recipient, is a further optional test of engraftment in the primary recipient. Engraftment can be detected by flow cytometry as 0.05% or greater human CD45+ cells in the blood, spleen or bone marrow at 1-4 months after administration of the human hematopoietic cells. A cytokine (e.g., GM-CSF) can be used to mobilize stem cells, for example, as described in Watanabe (1997, Bone Marrow Transplantation 19:1175-1181).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

The experiments presented herein were conducted to examine the clearance of red blood cells from the periphery and the mechanisms that mediate such a phenomenon. It is described herein that scavenger receptors play a role in human RBC sequestration and destruction, and that inhibition of scavenger receptor B1 can prevent RBC clearance.

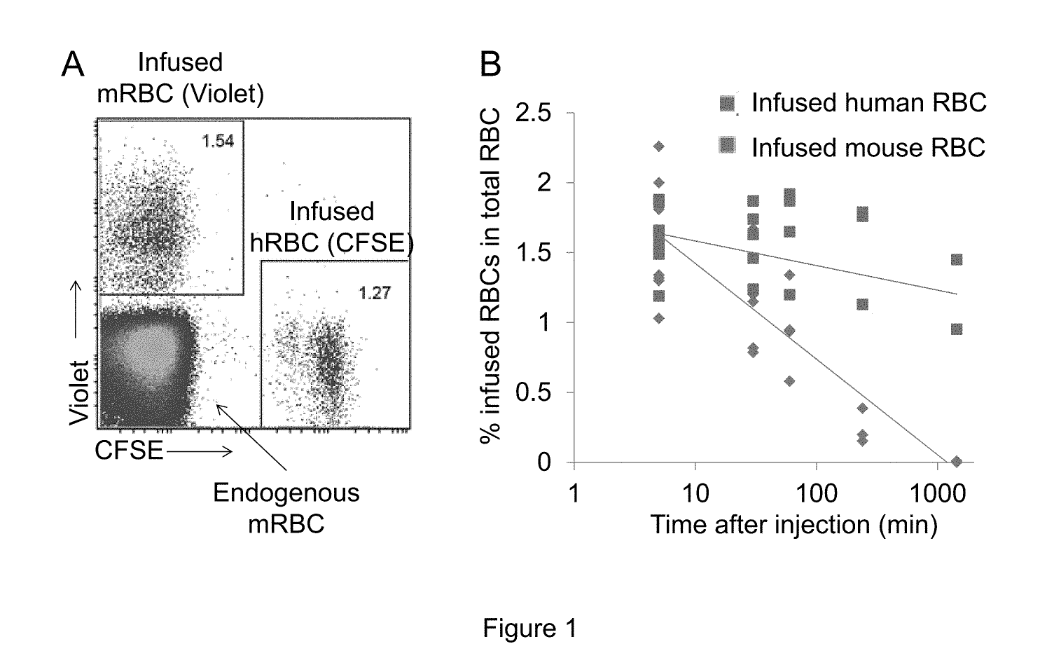

First, an experiment was conducted to examine the clearance of human red blood cells in mouse peripheral blood. Human and mouse red blood cells were pre-labeled with CFSE or violet dye, respectively, and then were transfused into RGSKI mice by retro-orbital injection. Peripheral blood was collected 5 minutes after blood infusion and the frequency of infused mouse or human RBCs was determined by FACS. It was observed that the frequency of infused human RBCs decreased more rapidly than mouse RBCs (FIG. 1B).

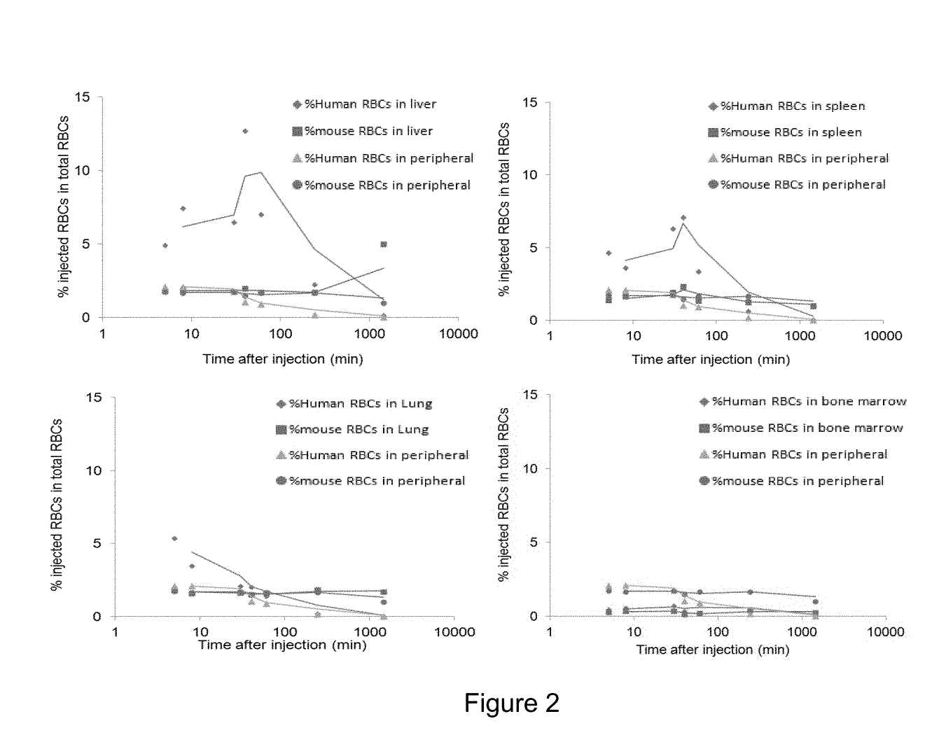

After infusion of human and mouse RBCs, the liver, lung, spleen, and bone marrow from the RGSKI mice were collected and the frequency in each tissue was analyzed as compared to frequency in the periphery (FIG. 2). It was found that infused human RBCs but not mouse RBCs were rapidly trapped in liver and spleen but not other tissues (FIG. 2).

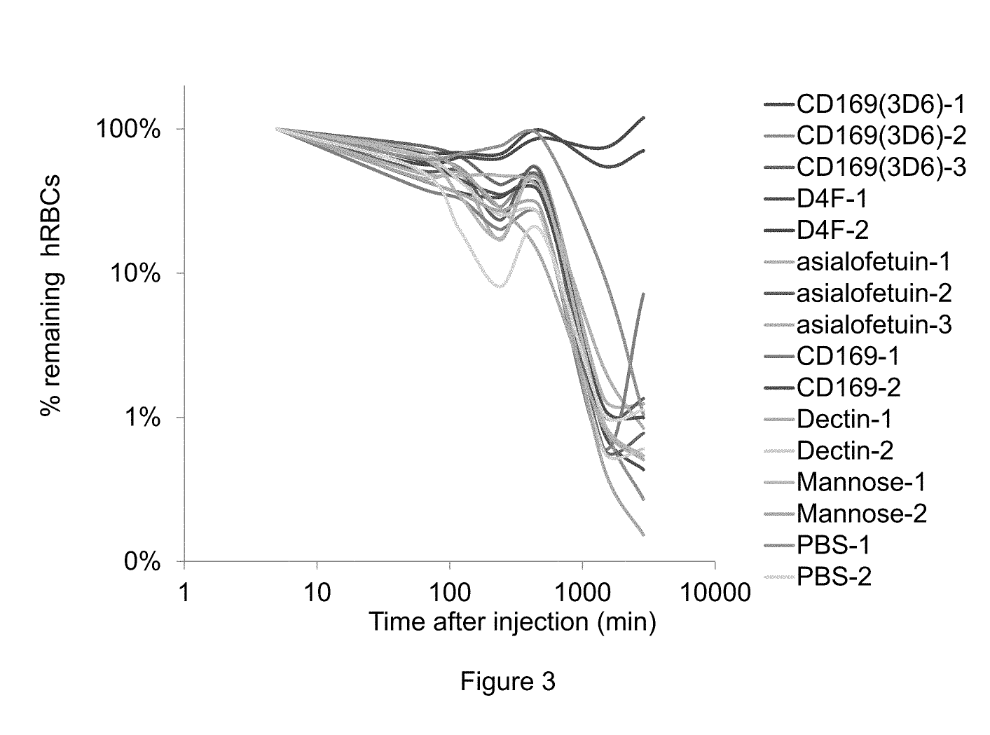

Experiments were then performed to evaluate the role of the scavenger receptor in RBC clearance. D-4F is an apolipoprotein A-I mimetic peptide and a general scavenger receptor inhibitor. D-4F is an 18 D-amino acid peptide (Ac-D-W-L-K-A-F-Y-D-K-V-A-E-K-L-K-E-A-F—NH₂; SEQ ID NO: 2). D-4F has been shown to increase cholesterol efflux and reduce inflammation and atherosclerosis (Navab et al., 2006, Nature Clinical Practice Cardiovascular Medicine, 3: 540-547). Other pathways which might involve in the destruction of human RBCs in the periphery were also tested. Inhibitors specific to different cell surface receptors that have been demonstrated to regulate red blood cell destruction were used, such as asialoglycoprotein receptor, sialic acid receptor, beta glucan receptor, etc. As shown in FIG. 3, only D4-F but not these other inhibitors protected infused human RBCs in the periphery, which demonstrates that scavenger receptor(s) are mainly responsible for the rapid destruction of human RBCs in the circulation.

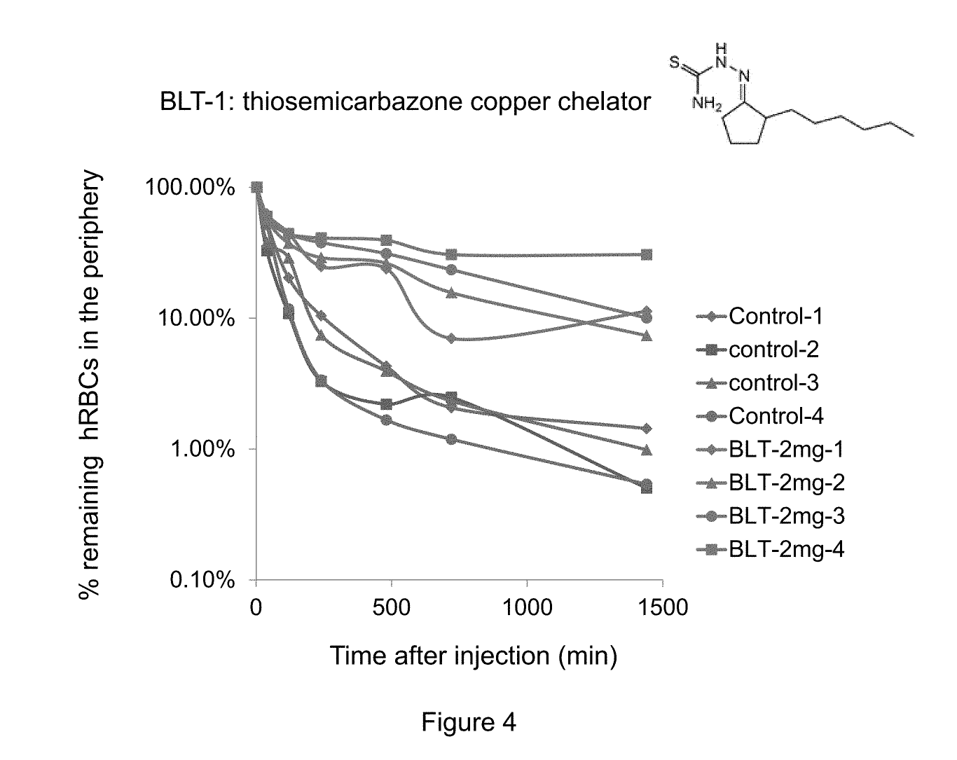

Further experiments were conducted to demonstrate that scavenger receptor B1 is implicated in human RBC destruction, and that inhibiting scavenger receptor B1 increases the frequency of human RBCs in the periphery (FIG. 4). As shown in FIG. 4, treatment of RGSKI mice with BLT-1 (C₁₂H₂₃N₃S; 2-Hexyl-1-cyclopentanone thiosemicarbazone) to block scavenger receptor B1 increases the percent of remaining RBCs in the periphery after human RBC infusion. This data demonstrates that inhibition of scavenger receptor B1 can inhibit or prevent human RBC destruction.

A targeted knockout of the receptor identified to be primarily responsible for human RBC destruction is generated. It is believed that, in some instances, genetically knocking out the receptor(s) will provide a more robust model than using blocking agents. Using the newly developed CRISPR/Cas system (Hale et al. 2012, Molecular Cell 45:292-302; Cong et al., 2013, Science 339:819-823; Wang et al., 2013, Cell 153:910-918), targeted mutation of particular scavenger receptor(s) is directly and rapidly introduced into mice.

The newly innovated CRISPR/Cas technology is used to generate scavenger receptor knockout mice on the RGSKI background. This technology has already enabled the production of multiple knockouts in a single step directly by injecting fertilized mouse eggs, dramatically shortening the time to generate these mice by a year or more. Normally transgenic mice are made by injecting fertilized eggs from standard mouse strains, commonly F2 mice. Because of the high efficiency of CRISPR and as cytoplasmic injection is used to make mice by CRISPR, this much quicker approach is used to generate fertilized eggs from the engraftment recipients. A similar colony (MTRG mice) has been used to successfully make blastocysts (a lower yield process than making fertilized eggs) and from them, germline transmitting ES cells. Moreover fertilized eggs were obtained and CRISPR technology was used in these eggs to make a different KO mouse in a triple KI mouse background. The new recipient mice have the following genotype:

Rag2^{-/-}Il2rg^{null/h}SIRPα^{h/h}/SR^{-/-} (RGSKI-SR^{-/-})

Further, RGSKI-SR−/− mice are generated which contain human hSIRPα, but do not carry any human cytokines that enhance HSC maintenance and erythropoiesis. Human RBC transfusion experiments are performed in RGSKI-SR^{-/-} mice and the decay of human RBCs is monitored in the periphery. In scavenger receptor knockout mice, prolonged maintenance of infused human RBCs in the peripheral blood is expected compared with control mice. Mice deficient in scavenger receptor achieve frequencies up to 50% human erythroid cells in the periphery similar to that in the bone marrow.

A scavenger receptor B1 knockout mouse was also developed on the MISTRG and MISTERG background. MISTERG contains the genes for human M-CSF, human IL-3/GM-CSF, human SIRPα, human TPO, and human EPO and does not express RAG2 or γ_{c}. MISTRG expresses human M-CSF, human IL-3/GM-CSF, human SIRPα, human TPO, and does not express RAG2 or γ_{c}.

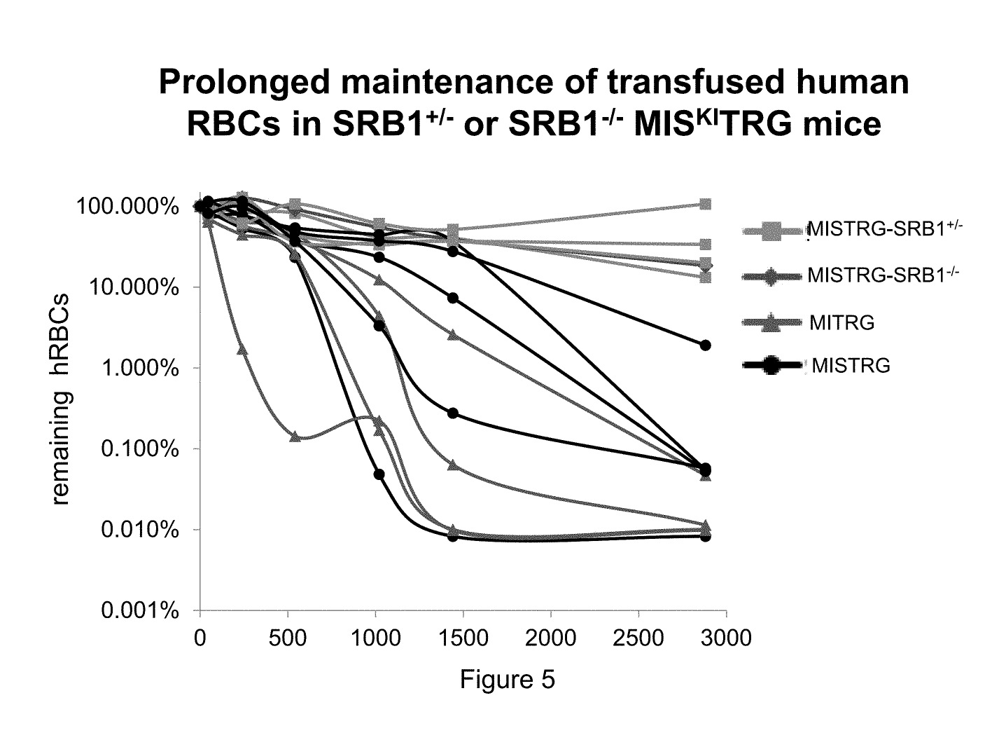

Experiments were conducted to evaluate the maintenance of human RBCs in scavenger receptor B1 knockout mice. MISTRG-SRB1^{-/-} or MISTRG-SRB1^{+/-} mice were infused with human RBCs and peripheral blood was collected at various time points. As shown in FIG. 5, it was observed that genetic depletion of scavenger receptor B1 protects infused human RBCs in the mouse system, as compared to control (MISTRG and MITRG mice).

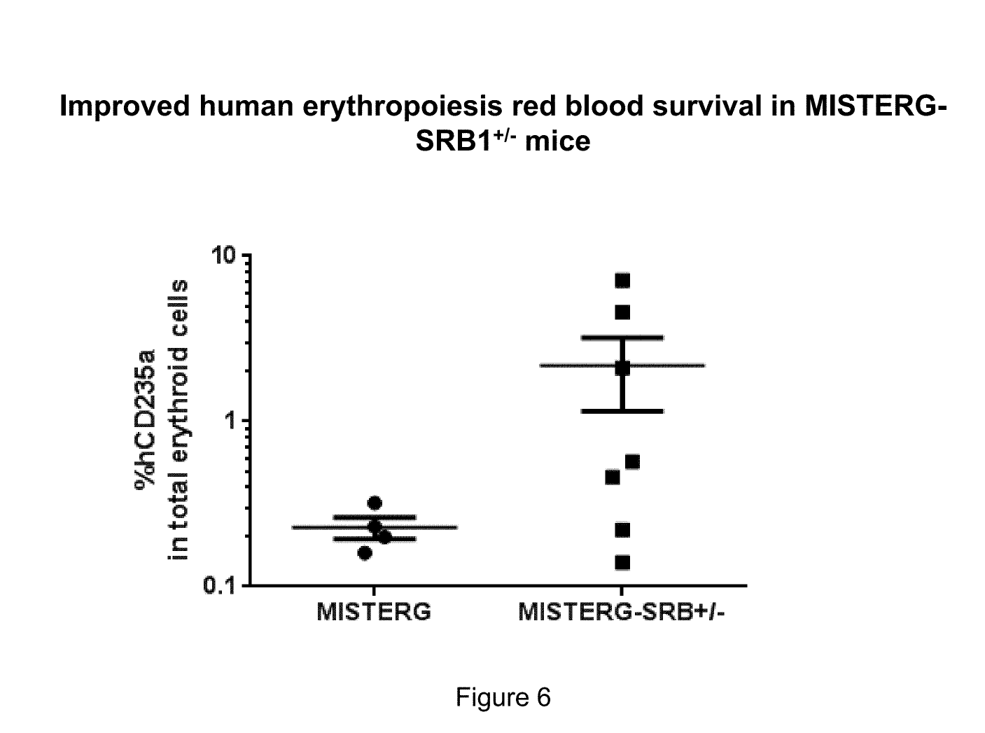

Experiments were also conducted to evaluate human erythropoiesis and RBC survival in scavenger receptor B1 knockout mice. MISTERG-SRB1^{+/-} mice were engrafted with human fetal liver CD34⁺ cells. 7 week post engraftment, mice were treated with clodronate and peripheral blood was collected for analysis. As shown in FIG. 6, genetic depletion of scavenger receptor B1 improves human erythropoiesis and red blood cell survival, as compared to control (MISTERG). Based on the drug inhibition studies already performed it is expected that blocking the scavenger receptor(s) significantly improves peripheral RBC numbers and survivability. More importantly, it is believed that further improvement of frequency of peripheral human erythroid cells could be achieved by specific inhibition or targeted mutation of scavenger receptor(s) identified.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for increasing red blood cells (RBCs) in peripheral blood comprising an inhibitor of one or more scavenger receptors.

2. The composition of claim 1, wherein the inhibitor is an inhibitor of scavenger receptor B1.

3. The composition of claim 1, wherein the inhibitor is selected from the group consisting of a nucleic acid, a siRNA, an antisense nucleic acid, a ribozyme, a peptide, a small molecule, an antagonist, an aptamer, and a peptidomimetic.

4. The composition of claim 1, wherein the inhibitor is selected from the group consisting of D-4F, BLT-1, and ITX-5061.

5. (canceled)

6. The composition of claim 1, wherein the composition reduces at least one selected from the group consisting of: the destruction of red blood cells and the sequestration of red blood cells.

7. (canceled)

8. A method for treating a subject in need thereof, the method comprising administering to the subject an effective amount of a composition comprising an inhibitor of one or more scavenger receptors, wherein the subject has or is at risk for developing at least one selected from the group consisting of: anemia and a cholesterol abnormality.

9. The method of claim 8, wherein the inhibitor is an inhibitor of scavenger receptor B1.

10. The method of claim 8, wherein the inhibitor is selected from the group consisting of a nucleic acid, a siRNA, an antisense nucleic acid, a ribozyme, a peptide, a small molecule, an antagonist, an aptamer, and a peptidomimetic.

11. The method of claim 8, wherein the inhibitor is selected from the group consisting of D-4F, BLT-1, and ITX-5061.

12. (canceled)

13. The method of claim 8, further comprising administering to the subject an effective amount of clodronate.

14. (canceled)

15. (canceled)

16. (canceled)

17. (canceled)

18. (canceled)

19. (canceled)

20. A genetically modified non-human animal comprising a genome that is modified such that the animal does not express one or more scavenger receptors.

21. The genetically modified animal of claim 20, wherein the one or more scavenger receptors comprise scavenger receptor B1.

22. The genetically modified animal of claim 20, comprising at least one of the group consisting of a nucleic acid encoding human EPO, a nucleic acid encoding human M-CSF, a nucleic acid encoding human IL-3, a nucleic acid encoding human GM-CSF, a nucleic acid encoding human SIRPα, and a nucleic acid encoding human TPO, wherein each of the nucleic acids encoding human M-CSF, human IL-3, human GM-CSF, human SIRPα, human TPO and human EPO is operably linked to a promoter, and wherein the animal expresses at least one of the group consisting of human EPO polypeptide, human M-CSF polypeptide, human IL-3 polypeptide, human GM-CSF polypeptide, human SIRPα polypeptide, and human TPO polypeptide.

23. The genetically modified non-human animal of claim 20, wherein the animal is immunodeficient.

24. The genetically modified immunodeficient non-human animal of claim 22, wherein the animal does not express recombination activating gene 2 (Rag-2−/−).

25. The genetically modified immunodeficient non-human animal of claim 22, wherein the animal does not express IL2 receptor gamma chain (gamma chain−/−).

26. The genetically modified immunodeficient non-human animal of claim 22, wherein the animal does not express Rag-2 and wherein the animal does not express IL2 receptor gamma chain (Rag-2−/− gamma chain−/−).

27. (canceled)

28. (canceled)

29. The genetically modified animal of claim 20, further comprising human hematopoietic cells.

30. The genetically modified animal of claim 20, further comprising human erythrocytes.

31. The genetically modified animal of claim 20, wherein the genetically modified animal is infected with malaria.

32. The genetically modified animal of claim 20, wherein the animal is treated with clodronate.


SEQUENCE LISTING (2 sequences)

SEQ ID NO: 1 - protein, 18 aa, Artificial Sequence
  Chemically Synthesized
DWLKAFYDKVAEKLKEAF

SEQ ID NO: 2 - protein, 18 aa, Artificial Sequence
  Chemically synthesized
  MISC_FEATURE (1)..(1): N-terminal acetylation
  MISC_FEATURE (18)..(18): C-terminal amidation
DWLKAFYDKVAEKLKEAF